(19) United States
(12) Patent Application Publication
(10) Pub. No.: US 2016/0374959 A1
(43) Pub. Date: Dec. 29, 2016

(54) TREATMENT OF EARLY STAGE PARKINSON'S DISEASE WITH A HYDROXYTYROSOL-CONTAINING POLYPHENOL FORMULATION

(71) Applicant: Allevium Therapeutics, Inc., Burlingame, CA (US)

(72) Inventor: Roberto Crea, Burlingame, CA (US)

(21) Appl. No.: 15/191,491

(22) Filed: Jun. 23, 2016

Related U.S. Application Data
(60) Provisional application No. 62/183,190, filed on Jun. 23, 2015.

(51) Int. Cl. A61K 31/05; A61K 9/48; A61K 9/08; A61K 45/06; A61K 31/7048; A61K 36/63; A61K 9/00; A61K 9/14

(52) U.S. Cl. CPC A61K 31/05; A61K 9/0053; A61K 9/48; A61K 9/14; A61K 45/06; A61K 31/7048; A61K 36/63; A61K 9/08

(57) ABSTRACT

The invention provides a method for treating a subject suffering from early stage Parkinson's disease with a pharmaceutical formulation containing an olive polyphenol composition. The olive polyphenol composition includes hydroxytyrosol and at least one additional olive polyphenol, and the hydroxytyrosol represents about 40 wt. % to about 90 wt. % of the olive polyphenol composition. The formulation is administered to the subject within the context of a dosing regimen that provides a daily dosage of the olive polyphenol composition in the range of 30 mg to about 2500 mg. The invention additionally provides a method for reducing the dose of an antiparkinsonism drug used in the treatment of early stage Parkinson's disease, as well as a pharmaceutical formulation for treating early stage Parkinson's disease.

### CROSS-REFERENCE TO RELATED APPLICATION

This application claims priority under 35 U.S.C. §119(e)(1) to provisional U.S. Patent Application Ser. 62/183,190, filed Jun. 23, 2015, the disclosure of which is incorporated by reference herein.

### TECHNICAL FIELD

The present invention relates generally to the treatment of neurodegenerative diseases, and more particularly relates to a method for treating a patient suffering from Parkinson's disease. The invention has utility in the fields of medicine, neurology, and pharmacotherapy.

### BACKGROUND

Parkinson's disease (PD) is the most common of the neurodegenerative movement disorders, predominantly involving cerebral dopaminergic neuronal circuits and resulting in progressive and irreversible motor dysfunction. While PD can affect people of any age, its incidence is age-related, and the lengthening life span of the world's population is causing an increase in the prevalence of neurodegenerative diseases such as PD. In the western world, PD currently affects approximately 1.5% to 2.0% of people aged 60 and older. PD is characterized by resting tremor, rigidity, bradykinesia or slowness, gait disturbance, and postural instability. See Olanow et al. (1999) Ann Rev Neurosci. 22:123-144.

In terms of pathology, PD is characterized by a progressive loss of dopaminergic neurons in the midbrain area, in a region known as the pars compacta of the substantia nigra. Symptoms appear when 50-80% of these neurons have died; see Mitchel, AJ, Neuropsychiatry and Behavioural Neurology Explained at 148-149 (London: Saunders, 2004). PD is also characterized by the presence of ubiquitin- and alpha-synuclein-positive cytoplasmic inclusions known as Lewy bodies, depigmentation of the locus ceruleus, and autonomic dysfunction including sympathetic denervation of the heart (Qi et al., cited above). Dopamine has thus been implicated as the endogenous neurotoxin to explain, at least in part, the selective neurodegeneration observed in PD.

Because loss of dopamine is now believed to be responsible for the majority of the motor symptoms of PD, treatment options have been primarily based upon restoration of dopamine function by replacement of dopamine precursors, inhibition of degradative enzymes, or dopamine agonists. Some efforts have also targeted the development of drugs for PD which are based on the synergistic action of dopamine, glutamate, and acetylcholine neurotransmission on GABAergic neurons in the striatum; see Qi et al., cited above. Other therapeutic interventions have focused on restoration of dopamine signaling; dopamine signaling, as is understood in the art, involves the storage, release, and recycling of dopamine in the presynaptic terminal and activation of pre- and post-synaptic receptors and various downstream signaling cascades.

Representative drugs used in the treatment of Parkinson's include: levodopa (or L-dopa), a precursor to various neurotransmitters, including dopamine; the dopamine receptor agonists apomorphine, pramipexole, ropinirole, and rotigotine; amantadine hydrochloride, an N-methyl-D-aspartate (NMDA) receptor antagonist; the monoamine oxidase B (MAO-B) inhibitors rasagiline and selegiline; the catechol-O-methyltransferase (COMT) inhibitors entacapone and tolcapone; and the antimuscarinic drugs benztropine mesylate, orphenadrine, procyclidine, and trihexyphenidyl.

Drugs used to treat PD themselves are associated with neuropsychiatric side effects. For example, dopamine agonists are well-known to cause sleep disturbance, dizziness, and even hallucinations, while antimuscarinic agents can cause confusion and impaired memory. L-dopa is generally viewed as the cornerstone of Parkinson's therapy, and helps with bradykinesia and rigidity. Its side effects, however, are numerous and severe.

There has also been considerable debate as to whether L-dopa may actually exacerbate PD due to the oxidation of L-dopa and its metabolites. There are many side effects of L-dopa, as noted above, including nausea, vomiting, hypotension, arrhythmias, disorientation and confusion, somnolence and narcolepsy, and severe dyskinesia (involuntary twisting and writhing). For this reason, L-dopa is generally not prescribed until a patient's Parkinson's disease has progressed to a late stage, and even then the dose typically has to be increased over time to maintain efficacy. There are currently no effective therapies for treating early stage Parkinson's patients and thus for preventing, postponing or delaying the neurodegenerative process.

It has recently been proposed that oxidative stress plays a role in the underlying mechanism that leads to the cellular dysfunction associated with PD. Oxidative stress occurs when a biological system is unable to readily detoxify reactive oxygen species (ROS) such as peroxides, the superoxide radical, the hydroxyl radical, and other free radicals formed as natural byproducts of the metabolism of oxygen. The resulting imbalance, i.e., the presence of excess ROS, causes a host of toxic effects in cellular components, including chemical and/or physical damage to proteins, lipids, and DNA (such as strand breaks and base damage). It is now believed that dopamine metabolism and associated processes contribute to oxidative stress. That is, the major sources of oxidative stress generated for the nigral dopaminergic neurons are thought to be the ROS produced during dopamine metabolism, mitochondrial dysfunction, and neuroinflammation; see Hwang (2013) Exp. Neurobiol. 22(1):11-17. It appears that the dopamine metabolite 3,4-dihydroxyphenylacetaldehyde (DOPAL), generated by intraneural deamination of dopamine by monoamine oxidase, is a particularly toxic agent, its toxicity occurring as the result of at least four distinct mechanisms, including cross-linking of proteins, oxidation to quinones, production of hydroxyl radicals, and exacerbation of the toxic effects of other agents. See Rees et al. (2009) Chem. Res. Toxicol. 22:1256-1263; Anderson et al. (2011) J. Biol. Chem. 286:26978-86; Li et al. (2001) Brain Res. Mol. Brain Res. 93:107; and Marchitti et al. (2007) Pharmacol. Rev. 59:125-150. To date, however, no pharmaceutical therapies for the treatment of PD have been developed that make use of the foregoing mechanism.

### SUMMARY OF THE INVENTION

Accordingly, the invention is directed to the aforementioned need in the art and provides a method for treating a subject suffering from early stage Parkinson's disease with an active agent formulation that is therapeutically effective in the treatment of early stage PD and thus in the prevention, delay, or slowing of the neurodegenerative process. The subject may be suffering from idiopathic or secondary Parkinson's disease. The method of treatment includes administering to the subject a therapeutically effective amount of a pharmaceutical formulation that comprises an olive polyphenol composition containing hydroxytyrosol and at least one additional olive polyphenol, wherein the hydroxytyrosol represents about 40 wt. % to about 90 wt. % of the olive polyphenol composition (also referred to as the total polyphenol composition, or “TPP”) and wherein the therapeutically effective amount provides a daily dose of the olive polyphenol composition in the range of 30 mg to about 2500 mg. Generally, the olive polyphenol composition represents on the order of about 5 wt. % to about 100 wt. % of the pharmaceutical formulation.

In another embodiment, the method of treatment involves oral administration of the pharmaceutical formulation to the subject, in which case the formulation will be composed of a suitable oral dosage form, such as a solution, suspension, tablet, capsule, powder, or the like. The oral dosage form will generally, although not necessarily, be a unit dosage form.

In a further embodiment, the method of treatment involves administration of a pharmaceutical formulation to the subject which is as described above but additionally provides for controlled release, e.g., sustained release, of the olive polyphenol composition.

In another embodiment, the method of treatment involves administration of the pharmaceutical formulation to the subject on the order of one to about six times daily to provide the daily dose indicated.

In still another embodiment, the method of treatment involves administration of a pharmaceutical formulation as above, but which includes co-administration of at least one additional active agent, e.g., an anti-parkinsonism agent such as L-dopa, a dopamine agonist, or the like. The additional active agent may be separately administered to the subject or incorporated into the pharmaceutical formulation and thus administered to the subject in a single composition.

The invention additionally provides a method for reducing the dosage of an anti-parkinsonism drug in the treatment of a subject suffering from early stage Parkinson's disease, where the method includes co-administering with the anti-parkinsonism drug a therapeutically effective amount of a pharmaceutical formulation that comprises an olive polyphenol composition containing hydroxytyrosol and at least one additional olive polyphenol, wherein the hydroxytyrosol represents about 40 wt. % to about 90 wt. % of the olive polyphenol composition, and the therapeutically effective amount provides a daily dose of the olive polyphenol composition in the range of 30 mg to about 2500 mg.

The invention also provides a pharmaceutical formulation for the treatment of early stage Parkinson's disease, where the formulation includes: (a) a therapeutically effective amount of an olive polyphenol composition containing hydroxytyrosol and at least one additional olive polyphenol, wherein the hydroxytyrosol represents about 40 wt. % to about 90 wt. % of the olive polyphenol composition; and (b) a therapeutically effective amount of an antiparkinsonism drug selected from L-dopa, dopamine receptor agonists, amantadine hydrochloride, monoamine oxidase B inhibitors, catechol-O-methyltransferase inhibitors; and antimuscarinic agents.

Additional objects, advantages, aspects, and novel features of the invention will be set forth in part in the description which follows, and in part will become apparent to those skilled in the art upon examination of the following, or may be learned by practice of the invention.

### DETAILED DESCRIPTION OF THE INVENTION

### Definitions and Overview

Unless defined otherwise, all technical and scientific terms used herein have the meaning commonly understood by one of ordinary skill in the art to which the invention pertains. Specific terminology of particular importance to the description of the present invention is defined below.

It must be noted that, as used in this specification and the appended claims, the singular forms “a,” “an” and “the” include plural referents unless the context clearly dictates otherwise. Thus, for example, “an active agent” refers not only to a single active agent but also to a combination of two or more different active agents, “a dosage form” refers to a combination of dosage forms as well as to a single dosage form, and the like.

Unless defined otherwise, all technical and scientific terms used herein have the meaning commonly understood by one of ordinary skill in the art to which the invention pertains. Specific terminology of particular importance to the description of the present invention is defined below. Unless defined otherwise, all technical and scientific terms used herein have the meaning commonly understood by one of ordinary skill in the art to which the invention pertains.

When referring to an active agent, applicants intend the term “active agent” to encompass not only the specified molecular entity but also its pharmaceutically acceptable, pharmacologically active analogs, including, but not limited to, salts, esters, amides, prodrugs, conjugates, active metabolites, crystalline forms (including polymorphs), enantiomers, and other such derivatives, analogs, and related compounds.

By the terms “effective amount” and “therapeutically effective amount” of a compound is meant a nontoxic but sufficient amount of an active agent to provide the desired effect, i.e., treatment of early stage Parkinson's disease.

“Treatment” of Parkinson's disease as the term is used herein refers to a reduction in the number of symptoms, a decrease in the severity of one or more symptoms, prevention of disease progression, delay of disease progression, and/or a decrease in the rate at which the disease progresses.

The term “unit dosage form” denotes any form of a pharmaceutical formulation that contains an amount of active agent sufficient to achieve a therapeutic effect with a single dose or single instance of administration. When the formulation is a tablet or capsule, the dosage form is usually one such tablet or capsule. The frequency of administration that will provide the most effective results in an efficient manner without overdosing will vary with the characteristics of the particular active agent, including both its pharmacological characteristics and its physical characteristics.

The term “controlled release” refers to a pharmaceutical formulation or fraction thereof in which release of the active agent is not immediate, i.e., with a “controlled release” formulation, administration does not result in immediate release of the active agent into an absorption pool. The term is used interchangeably with “nonimmediate release” as defined in Remington: The Science and Practice of Pharmacy, Nineteenth Ed. (Easton, Pa.: Mack Publishing Company, 1995). In general, the term “controlled release” as used herein includes sustained release and delayed release formulations.

The term “sustained release” (synonymous with “extended release”) is used in its conventional sense to refer to a pharmaceutical formulation that provides for gradual release of an active agent over an extended period of time, and that preferably, although not necessarily, results in substantially constant blood levels of the agent over an extended time period. The term “delayed release” is also used in its conventional sense, to refer to a pharmaceutical formulation that, following administration to a patient provides a measurable time delay before active agent is released from the formulation into the patient's body.

By “pharmaceutically acceptable” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration.

The invention thus pertains to a method for treating a subject having early stage Parkinson's disease by administering to the subject a formulation of olive polyphenols containing hydroxytyrosol and other olive polyphenols, with the ratio of hydroxytyrosol to other olive polyphenols in the formulation, dosage ranges, modes of administration, and other features of the inventive method described in detail below. The subject may have primary (idiopathic) Parkinson's disease, which may or may not involve a genetic origin, or the disease may be secondary, caused, for instance, by build-up of an ingested toxin or resulting from a more diffuse disease. The subject may also suffer from indeterminate parkinsonism, a relatively unusual situation in which it is not possible to determine whether the parkinsonism is primary or secondary.

Early Stage Parkinson's Disease:

A subject in “early stage Parkinson's disease” as that term is used herein has generally experienced a relatively recent onset of Parkinson's symptoms, i.e., onset within the previous fifteen years, typically within the previous nine years, and most usually within the previous six years. The subject has been diagnosed as having Parkinson's disease according to the clinical criteria established by the International Parkinson and Movement Disorder Society's (MDS) Task Force on Definition of Parkinson's Disease, described in Postuma et al. (2015) Movement Disorders 30(12).

A patient in the early stages of the disease is generally in the first, second, or third stages, preferably in the first or second stages, as those stages were initially defined by Hoehn and Yahr (Hoehn et al. (1967), “Parkinsonism: onset, progression and mortality,” Neurology 17(5):427-42). Hoehn and Yahr described five stages of the disease, as follows:

Stage one: This is the initial phase of the disease, in which an individual experiences relatively mild symptoms. These early symptoms generally include some or all of the following: tremors; a disturbance in gait; shaking in a limb; stiffness; slowness; muscle pain, cramps, or aching; and loss of dexterity. In this stage, the symptoms are not yet bilateral, and there is minimal or no functional impairment.

Stage two: In the second stage of Parkinson's disease, the individual's symptoms become bilateral, affecting both limbs and both sides of the body. There will usually be some difficulty walking and an increasing inability to perform ordinary physical tasks. Balance has not been impaired.

Stage three: Stage three of Parkinson's disease generally involves worsening of one or more of the symptoms experienced in Stages one and two, but also includes some degree of unsteadiness, involving impairment of balance and loss of equilibrium. There may also be some postural instability. The individual is still capable of living independently and may be able to work.

Stage four: In Stage four, the disease is fully developed and has become severely disabling. Although individuals in Stage four are still able to walk or stand unassisted, they are generally unable to perform day-to-day tasks and are markedly incapacitated. Additional symptoms experienced in Stage four includes rigidity and bradykinesia.

Stage five: In this final stage of Parkinson's disease, individuals become unable to walk or take care of themselves, and they are wheelchair-bound or bedridden unless assisted. A person in Stage five of the disease usually requires constant one-on-one nursing care.

The original Hoehn and Yahr staging scale was modified to include two additional stages, Stage 1.5, between Stages one and two above; and Stage 2.5, between Stages two and three above. In this modified scale, Stage 1.5 specifies that there is unilateral and axial involvement, in contrast to Stage one, where an individual experiences unilateral involvement only, and in contrast to Stage two, where an individual experiences bilateral involvement. Stage 2.5 of the modified Hoehn and Yahr scale specifies “mild bilateral disease with recovery on pull test”; see Goetz et al. (2004) Mov. Disord. 19:1020-1028. “Early stage” Parkinson's disease in the present context includes individuals in Stages one through three of the modified Hoehn and Yahr scale, typically individuals in Stages one, 1.5, two, and 2.5, and usually individuals in Stages one, 1.5, or 2.

An individual with “early stage” Parkinson's disease may also be diagnosed as such using the Unified Parkinson's Disease Rating Scale (UPDRS), a more recently established staging system that includes, in addition to the physical symptoms used in the Hoehn and Yahr scale, a number of additional criteria relating to non-motor aspects of daily living (nM-EDL) such as intellectual impairment, emotional states, motivation, and more. The UPDRS involves evaluation of of a total of 42 items and a rating system measured in ordinal levels, from 0 (no disability) to 199 (most severe). The UPDRS consists of four different parts: (1) Mentation, Behavior, and Mood; (2) Activities of Daily Living; (3) Motor Examination; and (4) Complications of Therapy (in the past week). Using the UPDRS criteria and scoring, an “early stage” Parkinson's patient for the purpose of the present invention indicates an individual with a UPDRS score in the range of up to about 75, typically up to about 50. See Fahn and Elton, “Unified Parkinson's Disease Rating Scale,” in Fahn et al., Recent Developments in Parkinson's Disease,” Vol. 2 (Florham Park, N.J.: MacMillan Healthcare Information, 1987), at pages 153-163 and 293-304.

The Olive Polyphenols:

Olive polyphenols have been shown to be potent anti-oxidant and anti-inflammatory compounds, and have been proposed for the purpose of bringing about a reduction in oxidative stress. Further, hydroxytyrosol has been confirmed to be an endogenous compound that is produced in the brain in small amounts during dopamine catabolism, and no toxicity has been detected even at high concentrations (Auñon-Calles et al. (2013) Food Chem. Toxicol. 55:498-504). That is, a pathway has been confirmed whereby monoamine oxidase catalyzes the oxidative deamination of dopamine to give the toxic dopamine metabolite DOPAL, which for the most part is oxidized by aldehyde dehydrogenase to give the carboxylic acid 3,4-dihydroxyphenylacetic acid (DOPAC), with a small portion of the DOPAL undergoing enzymatic reduction to yield hydroxytyrosol (Schröder et al. (2009) Am. J. Clin. Nutr. 90:1329-1335; Rodriguez-Morató et al. (2015) Molecules 20:4655-4680).

The bioavailability of the olive polyphenols, including hydroxytyrosol, has been established as quite good, on the order of 55-66 mol % or higher; see Vissers et al. (2004) Eur. Journal Clin. Nutrition 58:955-965. When the formulation is administered orally, the plasma Cₘₐₓ of hydroxytyrosol was found to be reached in only 13 minutes (i.e., the Tₘₐₓ); see Gonzalez-Santiago et al. (2010) Pharm. Res. 61:364-370. Furthermore, hydroxytyrosol is apparently one of the only polyphenols, if not the only polyphenol, that is able to cross the blood-brain barrier, i.e., the selectively permeable barrier separating circulating blood from the brain's extracellular fluid in the central nervous system. This in turn allows hydroxytyrosol to essentially penetrate throughout the central nervous system.

The inventor herein has found that purified hydroxytyrosol exerts a pro-oxidant effect in an endothelial cell based assay, but when combined with other olive polyphenols or minor components present in the vegetation water of olives, it results in a potent anti-oxidant. Without wishing to be bound by theory, it may be postulated that the removal of hydroxytyrosol from its original matrix and natural environment can result in adverse effects, and that at least some of the naturally occurring associated polyphenols are necessary to include in a therapeutic hydroxytyrosol formulation.

The “olive polyphenols” in the formulation are olive-derived polyphenols as described in U.S. Pat. Nos. 6,416,808, 7,216,909, 7,713,569, 8,216,599, and 8,263,142, all to Roberto Crea, the inventor herein. The disclosures of the aforementioned patents are incorporated by reference herein. As explained in the Crea patents, the olive-derived polyphenols include hydroxytyrosol and oleuropein as well as other polyphenols, and may be prepared from an olive-based starting material such as olives, pitted olives, olive oil, and olive pulp obtained from the manufacture of olive oil, which previously had been viewed as an olive oil by-product and treated as waste.

Conventionally, olive oil production involves crushing olives, including the pits, to produce a thick paste. During this procedure, the crushed olives are continuously washed with water, a process known as “malaxation.” The paste is then mechanically pressed to squeeze out the oil content. In addition to providing olive oil, the pressing also squeezes out the paste's water content. Such washing and pressing steps yield a considerable amount of water, referred to as “vegetation water.”

Both the pit and the pulp of olives are rich in water-soluble, phenolic compounds, including hydroxytyrosol, oleuropein, and other olive polyphenols. These compounds are extracted from olives during malaxation, according to their partition coefficients, and end up in the vegetation water. This explains why various phenolic compounds, such as oleuropein and its derivatives, produced in olive pulp, can be found in abundance in vegetation waters. The olives may be obtained from conventional and/or commercially available sources such as growers. Preferably, the vegetation water is obtained from pitted olives. To produce vegetation water, olive pulp from the olives is first pressed to obtain a liquid-phase mixture including olive oil, vegetation water, and solid by-products. Thereafter, the vegetation water is separated from the rest of the liquid phase mixture and collected. For purposes of commercial production, it may be desirable to automate various aspects of the invention, as described, for example, in U.S. Pat. Nos. 4,452,744, 4,522,119 and 4,370,274, each to Finch et al., incorporated herein by reference.

Hydroxytyrosol has a relatively simple molecular structure. It is a derivative of tyrosol, a phenethyl alcohol having the structure 4-(2-hydroxyethyl)phenol (also referred to as (p-hydroxyphenyl)ethanol, or p-HPEA) and also found in olive products. In hydroxytyrosol, the central benzene ring is substituted with an additional hydroxyl group ortho to the ring hydroxyl group present in tyrosol. Hydroxytyrosol is thus 4-(hydroxyethyl)-1,2-benzene diol (also referred to as (3,4-dihydroxyphenyl)ethanol or 3,4-DHPEA). Oleuropein is a glycosylated hydroxytyrosol ester of elenolic acid, and upon de-esterification, e.g., with acid, converts to hydroxytyrosol per se. The chemical structures of these compounds are as follows:

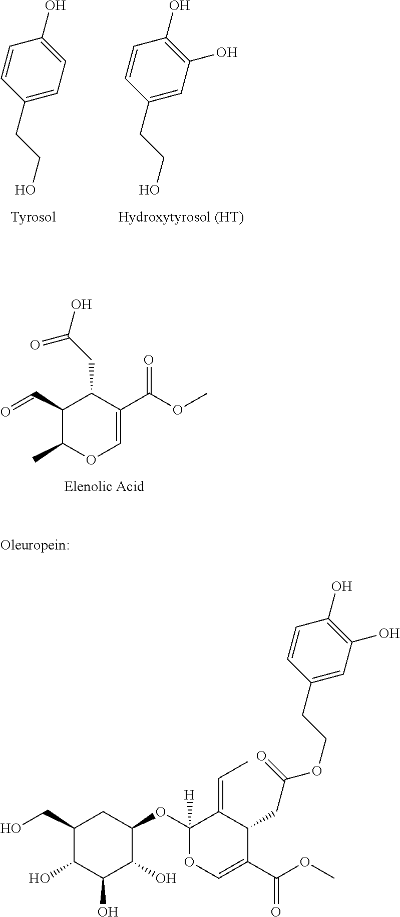 [SMILES: OCCc1ccc(O)cc1 ; OCCc1ccc(O)c(O)c1 ; COC(=O)C1=CO[C@@H](C)[C@@H](C=O)[C@@H]1CC(=O)O ; C/C=C1/[C@H](O[C@@H]2C[C@H](CO)[C@@H](O)[C@H](O)[C@H]2O)OC=C(C(=O)OC)[C@H]1CC(=O)OCCc1ccc(O)c(O)c1]

The polyphenols and their precursors found in olive products include, without limitation:

the phenolic alcohols hydroxytyrosol and tyrosol, the molecular structures of which are shown above, as well as other phenolic alcohols, including (3,4-dihydroxyphenyl)ethanol glucoside and 2-(4-hydroxyphenyl)ethyl acetate);

the benzoic acid derivatives gallic acid, gentisic acid, benzoic acid, vanillic acid, protocatecuic acid, p-hydroxybenzoic acid, and syringic acid;

the cinnamic acid derivatives caffeic acid, p-coumaric acid, o-coumaric acid, ferulic acid, cinnamic acid, and sinapinic acid;

other phenolic acids and derivatives including 4-(acetoxyethyl)-1,2-dihydroxybenzene, 3,4-dihydroxyphenylacetic acid (“DOPAC”), and 4-hydroxyphenylacetic acid;

the secoiridoids, characterized by the presence of either elenolic acid or elenolic acid derivatives in their molecular structure, including oleuropein, demethyloleuropein, 10-hydroxyoleuropein, oleuropein aglycone, oleuropein aglycone dialdehyde, ligstroside, 10-hydroxyligstroside, ligstroside aglycone, ligstroside aglycone dialdehyde, the dialdehydic form of decarboxymethyl elenolic acid linked to hydroxytyrosol, and the dialdehydic form of decarboxymethyl elenolic acid linked to tyrosol;

the hydroxy-isochromans 1-phenyl-6,7-dihydroxy-isochroman and 1-(3′-methoxy-4′-hydroxy)phenyl-6,7-dihydroxy-isochroman;

flavonoids including the flavones apigenin and luteolin, and the flavanonol (+)-taxifolin;

the lignans (+)-1-acetoxypinoresinol, (+)-pinoresinol, and (+)-1-hydroxypinoresinol.

The molecular structures of these compounds have been confirmed; see, e.g., Carrasco-Pancorbo et al. (2005) Journal of Separation Science 28(9-10).

Pharmaceutical Formulations, Dosage, and Modes of Administration:

In treating a subject with early stage Parkinson's disease as defined above, the invention involves administration of a therapeutically effective amount of a pharmaceutical formulation comprising an olive polyphenol composition that contains hydroxytyrosol in combination with at least one additional olive polyphenol, wherein the hydroxytyrosol represents 40 wt. % to about 90 wt. %, more typically about 40 wt. % to about 60 wt. %, and preferably about 42 wt. % to about 50 wt. % of the olive polyphenol composition, and wherein the therapeutically effective amount of the formulation administered provides a daily dose of the olive polyphenol composition in the range of 30 mg to about 2500 mg. Generally, the olive polyphenol composition represents on the order of about 5 wt. % to about 100 wt. % of the pharmaceutical formulation, typically on the order of about 5 wt. % to about 70 wt. % of the formulation, more typically on the order of about 10 wt. % to about 70 wt. % of the formulation, and most usually about 10 wt. % to about 30 wt. % of the formulation, with an inert filler representing any non-polyphenol portion of the formulation, the filler being, for instance, a carrier containing one or more inert excipients as will be described infra.

The at least one additional olive polyphenol generally includes oleuropein, the hydroxytyrosol ester whose molecular structure is provided above. In this embodiment, the weight ratio of hydroxytyrosol to oleuropein in the olive polyphenol composition (and thus in the pharmaceutical formulation) is in the range of about 1:1 to about 1:100, typically, although not necessarily, in the range of about 1:1 to about 9:1.

When the formulation is orally administered to the patient, an appropriate daily dose is in the range of 30 mg to about 2500 mg total polyphenols, typically in the range of 35 mg to about 1000 mg total polyphenols, and usually in the range of 35 mg to 300 mg total polyphenols. Examples of suitable daily doses of total polyphenols thus include, without limitation, 30 mg, 35 mg, 50 mg, 125 mg, 144 mg, 175 mg, 216 mg, 500 mg, 750 mg, 1000 mg, 1500 mg, and 2000 mg, and examples of suitable daily dose ranges of total polyphenols thus include, without limitation, 30 mg to 2000 mg, 30 mg to 500 mg, 30 mg to 300 mg, 35 mg to 1000 mg, 35 mg to 500 mg, 50 mg to 350 mg, and 70 mg to 325 mg. A daily dose of 30 mg to 2500 mg, is equivalent to about 0.2 mg/kg/day to about 50 mg/kg/day (corresponding to a patient weight range of 50 kg to 150 kg), and for the average person weighing in the range of about 60 kg to 80 kg, corresponds to about 0.4 mg/kg/day to about 41.7 mg/kg/day. Similarly, it will be appreciated that a daily dose of 30 mg to 1000 mg, is equivalent to about 0.2 mg/kg/day to about 20 mg/kg/day (again for a patient weight range of 50 kg to 150 kg), and for the average person weighing in the range of about 60 kg to 80 kg, corresponds to about 0.4 mg/kg/day to about 16.7 mg/kg/day. Analogously, a daily dose of polyphenols in the range of 35 mg to 300 mg corresponds to about 0.2 mg/kg/day to about 6.0 mg/kg/day (as before, for a patient weight range of 50 kg to 150 kg), and, similarly, for the average person weighing in the range of about 60 kg to 80 kg, corresponds to about 0.4 mg/kg/day to about 5.0 mg/kg/day. For ease of understanding, these conversions are summarized in Table 1. Table 2 shows the corresponding daily doses of hydroxytyrosol per se (i.e., not including other polyphenols), calculated at four different wt. % values (i.e., wt. % HT in the total polyphenols):

TABLE 1

| | | |
|---|---|---|
| Daily Dose of Total | Patient | Total Polyphenol Dose |
| Polyphenols, mg | weight, kg | Converted to mg/kg/day* |

| | | | | | |
|---|---|---|---|---|---|
| 30 | mg | 50 | kg | 0.6 | mg/kg/day |
| 30 | mg | 60 | kg | 0.5 | mg/kg/day |
| 30 | mg | 80 | kg | 0.4 | mg/kg/day |
| 30 | mg | 150 | kg | 0.2 | mg/kg/day |
| 35 | mg | 50 | kg | 0.7 | mg/kg/day |
| 35 | mg | 60 | kg | 0.6 | mg/kg/day |
| 35 | mg | 80 | kg | 0.4 | mg/kg/day |
| 35 | mg | 150 | kg | 0.2 | mg/kg/day |
| 300 | mg | 50 | kg | 6.0 | mg/kg/day |
| 300 | mg | 60 | kg | 5.0 | mg/kg/day |
| 300 | mg | 80 | kg | 3.8 | mg/kg/day |
| 300 | mg | 150 | kg | 2.0 | mg/kg/day |
| 1000 | mg | 50 | kg | 20.0 | mg/kg/day |
| 1000 | mg | 60 | kg | 16.7 | mg/kg/day |
| 1000 | mg | 80 | kg | 12.5 | mg/kg/day |
| 1000 | mg | 150 | kg | 6.7 | mg/kg/day |
| 2500 | mg | 50 | kg | 50.0 | mg/kg/day |
| 2500 | mg | 60 | kg | 41.7 | mg/kg/day |
| 2500 | mg | 80 | kg | 31.3 | mg/kg/day |
| 2500 | mg | 150 | kg | 16.7 | mg/kg/day |

*Dose in mg/kg/day rounded to the nearest tenth.

TABLE 2

| | | Daily dose | | | | | | | | | |
|---|---|---|---|---|---|---|---|---|---|---|---|
| Daily Dose | | HT, 33.3 | | Daily dose | | Daily dose | | Daily dose | | Daily dose | |
| of Polyphenols** | | wt %*** | | HT, 38 wt % | | HT, 42 wt % | | HT, 46 wt % | | HT, 50 wt % | |
| 30 | mg | 10.0 | mg | 11.4 | mg | 12.6 | mg | 13.8 | mg | 15.0 | mg |
| 30 | mg | 10.0 | mg | 11.4 | mg | 12.6 | mg | 13.8 | mg | 15.0 | mg |
| 30 | mg | 10.0 | mg | 11.4 | mg | 12.6 | mg | 13.8 | mg | 15.0 | mg |
| 30 | mg | 10.0 | mg | 11.4 | mg | 12.6 | mg | 13.8 | mg | 15.0 | mg |
| 35 | mg | 11.7 | mg | 13.3 | mg | 14.7 | mg | 16.1 | mg | 17.5 | mg |
| 35 | mg | 11.7 | mg | 13.3 | mg | 14.7 | mg | 16.1 | mg | 17.5 | mg |
| 35 | mg | 11.7 | mg | 13.3 | mg | 14.7 | mg | 16.1 | mg | 17.5 | mg |
| 35 | mg | 11.7 | mg | 13.3 | mg | 14.7 | mg | 16.1 | mg | 17.5 | mg |
| 300 | mg | 100 | mg | 114.0 | mg | 126.0 | mg | 138.0 | mg | 150.0 | mg |
| 300 | mg | 100 | mg | 114.0 | mg | 126.0 | mg | 138.0 | mg | 150.0 | mg |
| 300 | mg | 100 | mg | 114.0 | mg | 126.0 | mg | 138.0 | mg | 150.0 | mg |
| 300 | mg | 100 | mg | 114.0 | mg | 126.0 | mg | 138.0 | mg | 150.0 | mg |
| 1000 | mg | 333.0 | mg | 380.0 | mg | 420.0 | mg | 460.0 | mg | 500.0 | mg |
| 1000 | mg | 333.0 | mg | 380.0 | mg | 420.0 | mg | 460.0 | mg | 500.0 | mg |
| 1000 | mg | 333.0 | mg | 380.0 | mg | 420.0 | mg | 460.0 | mg | 500.0 | mg |
| 1000 | mg | 333.0 | mg | 380.0 | mg | 420.0 | mg | 460.0 | mg | 500.0 | mg |
| 2500 | mg | 832.5 | mg | 950.0 | mg | 1050.0 | mg | 1150.0 | mg | 1250.0 | mg |
| 2500 | mg | 832.5 | mg | 950.0 | mg | 1050.0 | mg | 1150.0 | mg | 1250.0 | mg |
| 2500 | mg | 832.5 | mg | 950.0 | mg | 1050.0 | mg | 1150.0 | mg | 1250.0 | mg |
| 2500 | mg | 832.5 | mg | 950.0 | mg | 1050.0 | mg | 1150.0 | mg | 1250.0 | mg |

**This is the daily dose of total polyphenols.
***This is the daily dose of HT when HT represents 33.3 wt. % of the total polyphenols in the formulation.

Although the dosages above are given as daily dosages, it is to be understood that administration does not need to be once daily. The formulation may be administered anywhere from one to about six times a day, and is typically administered two to six times a day, e.g., two to four times a day, three to six times a day, or the like. Further, while the Parkinson's subject generally receives the formulation at least once a day, administration every day, i.e., at least once every day, is highly preferred for maximum efficacy but it is not necessarily an essential feature of the present method.

As discussed above, the pharmaceutical formulation administered in the method of the invention comprises an olive polyphenol composition containing hydroxytyrosol and at least one other olive polyphenol; generally, the at least one other olive polyphenol includes oleuropein, a complex hydroxytyrosol ester. The relative amount of hydroxytyrosol in the olive polyphenol composition is discussed above, i.e., the hydroxytyrosol generally represents about 40 wt. % to about 90 wt. % of the olive polyphenol composition, typically about 40 wt. % to about 60 wt. % of the olive polyphenol composition, and preferably about 42 wt. % to about 50 wt. % of the olive polyphenol composition; this can be ensured by the incorporation of an acid in the formulation. The acid serves to de-esterify the oleuropein as well as any other elenolic acid esters (or other esters) that may be present. The acid may be an inorganic acid such as, for example, hydrochloric, sulfuric, or phosphoric acid, but is preferably an organic acid such as citric acid, acetic acid, oxalic acid, or the like, with citric acid being generally preferred. The amount of acid added is selected to generate a ratio of hydroxytyrosol to other polyphenols, e.g., oleuropein, in the ratio ranges given above. Typically, the amount of acid added provides the formulation with a pH in the range of about 2 to about 4.5, more typically in the range of about 2.5 to about 4.0. As an example, in a scaled-up manufacturing process, solid citric acid can be added while continuously stirring in an amount of preferably about 25 to 50 pounds of acid per about 1000 liters of vegetation water.

If the formulation is to be maintained in liquid form for pharmaceutical use, particularly for oral administration, a liquid carrier may be used, which is generally although not necessarily an aqueous carrier, in an amount effective to provide the desired concentration of the olive polyphenol composition. The aqueous vegetation water obtained in the method for obtaining olive polyphenols described earlier in this section may be used as is, or may be concentrated to a desired extent or modified with additives that confer a beneficial effect without adversely affecting the formulation. The concentration of the olive polyphenol composition in the liquid pharmaceutical formulation is generally in the range of about 5 wt. % to about 100 wt. %, typically in the range of about 10 wt. % to about 70 wt. %.

Other suitable carriers for liquid pharmaceutical formulations used herein include saline and other salt solutions, alcohols, vegetable oils, preservatives, buffer systems, colorants, flavoring agents, and other such substances that do not adverse affect the active agents, the formulation, or therapeutic efficacy.

Administration of the formulation in carrying out the method of the invention generally involves oral administration, but other modes of administration may be used as well. Thus, administration can be, for example, oral, parenteral, transdermal, transmucosal (including rectal and vaginal), sublingual, by inhalation, or via an implanted reservoir in a dosage form. The term “parenteral” as used herein is intended to include subcutaneous, intravenous, and intramuscular injection. In any of these pharmaceutical formulations, the percentage of hydroxytyrosol to total polyphenols in the olive phenol composition will be the same as those given above, i.e., generally in the range of about 40 wt % to about 90 wt. %, typically in the range of about 40 wt. % to about 60 wt. %, and preferably in the range of about 42 wt. % to about 50 wt. %.

Depending on the intended mode of administration, the pharmaceutical formulation may be a solid, semi-solid or liquid, such as, for example, a tablet, a capsule, a caplet, a liquid, a suspension, an emulsion, a suppository, granules, pellets, beads, a powder, or the like, preferably in unit dosage form suitable for single administration of a precise dosage. Suitable pharmaceutical compositions and dosage forms may be prepared using conventional methods known to those in the field of pharmaceutical formulation and described in the pertinent texts and literature, e.g., in Remington: The Science and Practice of Pharmacy (Easton, Pa.: Mack Publishing Co., 1995).

Oral dosage forms may be tablets, capsules, or caplets, or liquid formulations such as solutions, suspensions and syrups. They may also comprise a plurality of granules, beads, powders, or pellets that may or may not be encapsulated.

Tablets may be manufactured using standard tablet processing procedures and equipment. Direct compression and granulation techniques are preferred. In addition to the active agent, tablets may contain inactive, pharmaceutically acceptable carrier materials such as binders, lubricants, disintegrants, fillers, stabilizers, surfactants, coloring agents, and the like.

Capsules are also preferred oral dosage forms, in which case the active agent-containing formulation may be encapsulated in the form of a liquid or solid (including particulates such as granules, beads, powders or pellets). Suitable capsules may be either hard or soft, and are generally made of gelatin, starch, or a cellulosic material, with gelatin capsules preferred. Two-piece hard gelatin capsules are preferably sealed, such as with gelatin bands or the like. See, for example, Remington: The Science and Practice of Pharmacy, cited supra, which describes materials and methods for preparing encapsulated pharmaceuticals.

Oral dosage forms, whether tablets, capsules, caplets, or particulates, may, if desired, be formulated as controlled release preparations, so as to provide for delayed release of the active agents and/or for gradual, sustained release of the active agents over an extended time period. Generally, as will be appreciated by those of ordinary skill in the art, controlled release dosage forms may be formulated by dispersing the active agent within a matrix of a gradually hydrolyzable material such as a hydrophilic polymer, or by coating a solid, drug-containing dosage form with such a material. Hydrophilic polymers useful for providing a sustained release coating or matrix include, by way of example: cellulosic polymers such as hydroxypropyl cellulose, hydroxyethyl cellulose, hydroxypropyl methyl cellulose, methyl cellulose, ethyl cellulose, cellulose acetate, and carboxymethylcellulose sodium; acrylic acid polymers and copolymers, preferably formed from acrylic acid, methacrylic acid, acrylic acid alkyl esters, methacrylic acid alkyl esters, and the like, e.g. copolymers of acrylic acid, methacrylic acid, methyl acrylate, ethyl acrylate, methyl methacrylate and/or ethyl methacrylate; and vinyl polymers and copolymers such as polyvinyl pyrrolidone, polyvinyl acetate, and ethylene-vinyl acetate copolymer.

Commercial products are available for use in the present method. Exemplary products are available under the trade names Olivenol® and Olivenol Plus®, both formulated with Hidrox®, an olive polyphenol composition in the form of an Olea europaea extract containing hydroxytyrosol and other olive polyphenols, with the hydroxytyrosol representing about 43-45 wt. % of the total olive polyphenols. Hidrox 6% contains 6 wt. % total polyphenols, with the remainder being composed primarily of naturally occurring materials or derivatives thereof, like carbohydrates, some lipids, and small amounts of proteins, and Hidrox 12% contains 12 wt. % olive polyphenols in an analogous composition, but with suspended particles having been removed by ultracentrifugation. Olivenol Plus capsules contain 200 mg of Hidrox 6% plus an inert excipient as a filler, such that administration of two Olivenol Plus capsules to a patient provides a dose of 24 mg olive polyphenols, with hydroxytyrosol representing about 11 mg of that amount, while administration of six Olivenol Plus capsules to a patient provides a dose of 72 mg olive polyphenols, with hydroxytyrosol representing about 33 mg of that amount. Another product that can be used in the present method is AlleOne™, containing 300 mg Hidrox 12% in combination with rice starch as a filler. Other such products may be formulated similarly, containing, for instance, 100 to 500 mg of Hidrox 6% or Hidrox 12%. In addition to capsules, Olivenol products are available as liquid formulations and as freeze-dried powders.

Preparations according to this invention for parenteral administration include sterile aqueous and nonaqueous solutions, suspensions, and emulsions. Parenteral formulations may contain adjuvants such as solubilizers, preservatives, wetting agents, emulsifiers, dispersants, and stabilizers, and aqueous suspensions may contain substances that increase the viscosity of the suspension, such as sodium carboxymethyl cellulose, sorbitol, and dextran. Injectable formulations are rendered sterile by incorporation of a sterilizing agent, filtration through a bacteria-retaining filter, irradiation, or heat. They can also be manufactured using a sterile injectable medium. The active agent may also be in dried, e.g., lyophilized, form that may be rehydrated with a suitable vehicle immediately prior to administration via injection.

The method of the invention may involve administration of the pharmaceutical formulation through the skin or mucosal tissue using transdermal or transmucosal drug delivery systems. In transdermal drug delivery systems, as is known in the art, the active agent is contained within a laminated structure that serves as a drug delivery device to be affixed to the skin. In such a structure, the active agent-containing formulation may be contained in a layer, or “reservoir,” underlying an upper backing layer. The laminated structure may contain a single reservoir, or it may contain multiple reservoirs. In one embodiment, the reservoir comprises a polymeric matrix of a pharmaceutically acceptable contact adhesive material that serves to affix the system to the skin during drug delivery. Alternatively, the active agent-containing reservoir and skin contact adhesive are present as separate and distinct layers, with the adhesive underlying the reservoir which, in this case, may be either a polymeric matrix as described above, or it may be a liquid or hydrogel reservoir, or may take some other form. Transdermal drug delivery systems may in addition contain a skin permeation enhancer.

In addition, the pharmaceutical formulation described herein may also be formulated as a depot preparation for controlled release of the active agents, preferably sustained release over an extended time period. These sustained release dosage forms are generally administered by implantation (e.g., subcutaneously or intramuscularly or by intramuscular injection).

Other modes of administration are suitable as well. For example, administration may be rectal or vaginal, preferably using a suppository that contains, in addition to the active agents, excipients such as a suppository wax. Formulations for nasal or sublingual administration can also be prepared, using standard excipients known in the art. The active agents may also be formulated for inhalation, e.g., as a solution in saline, as a dry powder, or as an aerosol.

Co-Administration with Other Active Agents:

It may be advantageous in some cases, e.g., with a patient who has not responded sufficiently to the initial therapy with the pharmaceutical formulations described above, or who begins treatment in a somewhat advanced point in early Stage Parkinson's (such as in Stages 2.5 or 3), to co-administer at least one additional active agent along with the olive polyphenol formulation. The at least one additional active agent is preferably an anti-parkinsonism drug and may be selected from, for example, L-dopa; a dopamine agonist, (e.g., apomorphine, pramipexole, ropinirole, rotigotine, or the like; dopamine or another neurotransmitter; an N-methyl-D-aspartate (NMDA) receptor antagonist such as memantine; rasagiline, selegiline, or other MAO-B inhibitors; the catechol-O-methyltransferase (COMT) inhibitors entacapone and tolcapone; or an antimuscarinic drug such as benztropine mesylate, orphenadrine, procyclidine, and trihexyphenidyl. Of these, L-dopa and the dopamine agonists are generally preferred.

A significant advantage of co-administering the polyphenol formulation discussed hereinabove with at least one additional active agent in the form of an anti-parkinsonism drug such as L-dopa or a dopamine agonist is that the side effects encountered with L-dopa or dopamine agonist monotherapy can be reduced, because co-administration with the olive polyphenol composition enables a reduction in dosage of the additional agent. The dosage of the additional active agent, e.g., L-dopa or a dopamine agonist, is reduced by at least 5%, preferably at least 15%, and optimally at least 50%, relative to the prescribed dosage when the additional active agent is administered alone. The at least one additional active agent may be administered at the same time as the olive polyphenol formulation, or it may be administered at a different time. If the at least one additional active agent and the olive polyphenol composition are administered simultaneously, they may be co-administered separately, i.e., in two discrete dosage forms, or they may be combined into a single dosage form.

In a related aspect of the invention, a method is provided for reducing the dosage of an anti-parkinsonism drug in the treatment of a subject suffering from early stage Parkinson's disease, the method including co-administering with the anti-parkinsonism drug a therapeutically effective amount of a pharmaceutical formulation that comprises: an olive polyphenol composition containing hydroxytyrosol and at least one other olive polyphenol, wherein the hydroxytyrosol represents about 40 wt. % to about 90 wt. % of the olive polyphenol composition, typically about 42 wt. % to about 50 wt. % of the olive polyphenol composition, and wherein the therapeutically effective amount provides a daily dose of the olive polyphenol composition in the range of 30 mg to about 2500 mg, with typical subsets of this range given earlier herein.

In another embodiment of the invention, a pharmaceutical formulation is provided for the treatment of a subject suffering from early stage Parkinson's disease, where the pharmaceutical formulation comprises an olive polyphenol composition containing hydroxytyrosol and at least one other olive polyphenol, wherein the hydroxytyrosol represents about 40 wt. % to about 90 wt. % of the olive polyphenol composition, typically about 42 wt. % to about 50 wt. % of the olive polyphenol composition, and additionally contains a therapeutically effective amount of an anti-parkinsonism drug. The anti-parkinsonism drug is generally selected from those described supra and thus include L-dopa; a dopamine agonist, e.g., apomorphine, pramipexole, ropinirole, rotigotine, or the like; dopamine or another neurotransmitter; an N-methyl-D-aspartate (NMDA) receptor antagonist such as memantine; rasagiline, selegiline, or other MAO-B inhibitors; the catechol-O-methyltransferase (COMT) inhibitors entacapone and tolcapone; or an antimuscarinic drug such as benztropine mesylate, orphenadrine, procyclidine, and trihexyphenidyl. Of these, L-dopa and the dopamine agonists are generally preferred.

The formulation is generally, although not necessarily, prepared as a unit dosage form, with each dosage form providing a single dose of the olive polyphenol composition, or fraction such as half, one-third, or one-quarter of a single dose of the olive polyphenol composition, in which case two, three, or four such unit dosage forms (respectively) when combined provide the single dose. The single dose is in the range of about 30 mg to about 2500 mg, typically in the range of about 35 mg to about 1000 mg, most usually in the range of about 35 mg to about 300 mg. The at least one other olive polyphenol generally, and preferably, includes oleuropein, in which case the weight ratio of hydroxytyrosol to oleuropein is normally in the range of about 1:1 to about 100:1, more typically in the range of about 1:1 to about 9:1. Suitable dosage forms are described earlier herein, and include, by way of example, orally administrable liquid or solid formulations.

In an additional embodiment, a method is provided for treating a subject suffering from Parkinson's disease, either idiopathic or secondary, with a therapeutically effective amount of a pharmaceutical formulation that comprises an olive polyphenol composition containing hydroxytyrosol and at least one additional olive polyphenol, wherein the hydroxytyrosol represents about 40 wt. % to about 90 wt. % of the olive polyphenol composition, preferably about 42 wt. % to about 50 wt. % of the olive polyphenol composition, and wherein the therapeutically effective amount provides a daily dose of the olive polyphenol composition in the range of 30 mg to about 2500 mg, typically 35 mg to about 100 mg, preferably about 35 mg to about 300 mg. Generally, the olive polyphenol composition represents on the order of about 3 wt. % to about 100 wt. % of the pharmaceutical formulation. The pharmaceutical formulation may include a carrier, in which case the olive polyphenol composition represents about 2.5 wt. % to about 99 wt. % of the formulation.

In the aforementioned embodiment, an anti-parkinsonism drug such as L-dopa, a dopamine agonist, or the like, may be co-administered with the olive polyphenol composition as described above.

The invention thus provides an effective treatment for early stage Parkinson's disease, by administration of a formulation containing at least one active agent that crosses the blood-brain barrier, exhibits good bioavailability, is endogenous, beneficially affects the dopamine pathway to reduce ROS, and reduces, prevents, and/or counteracts neuroinflammation in the early stages of the disease. The method delays, prevents, or slows the progression of Parkinson's disease in patients in the early stages of the disease. The method can also be adapted to prevent the onset of Parkinson's disease in patients who are genetically predisposed to develop it. This is a significant advance in the treatment of a widespread, debilitating neurodegenerative disease for which no adequate therapy has been developed.

It is to be understood that while the invention has been described in conjunction with a number of specific embodiments, the foregoing description as well as the examples that follow are intended to illustrate and not limit the scope of the invention. Other aspects, advantages and modifications will be apparent to those skilled in the art.

### Example 1

A female subject 45 years of age was diagnosed with Parkinson's disease less than twelve months prior to beginning treatment as described in this example, and had been treated with orally administered L-dopa for over three months prior to beginning treatment with the present composition. There was no noticeable improvement after treatment with L-dopa, and the patient had begun to exhibit symptoms of muscle rigidity and muscle weakness. She was unable to stand on one foot, could not spread her toes, and exhibited tremors in her hands and legs.

The subject was given an olive polyphenol composition orally, in the form of two Olivenol Plus® capsules administered three times a day. As each capsule contains 12 mg olive polyphenols, including about 5 mg hydroxytyrosol, the daily dose given to the subject was 72 mg olive polyphenols, including 30 mg hydroxytyrosol. After just one week of treatment, the subject began experiencing a dramatic improvement in her conditions. She noticed that when playing the piano, she experienced a 25% reduction in leg tremor. She was also able to balance on one foot and spread her toes. There was an overall 50% improvement in isolation of movement on her left side, and the standard physical tests used in evaluation of parkinsonism, including metronome pacing, were back in the normal range.

### Example 2

This example describes an eight-week pilot open-label crossover interventional study (“OLI-PD”) that follows 17 PD patients receiving their current medication as well as their current medication and Olivenol Plus® (two capsules, administered tid), each containing 72 mg olive polyphenols, including about 33 mg hydroxytyrosol. The study is taking place in the Neurology and Research Departments of Colentina Clinical Hospital (Bucharest, Romania). The Olivenol Plus is provided by Dr. Roberto Crea, the inventor herein and president of Allevium Therapeutics Inc., the assignee of this patent application.

OLI-PD is making use of data from two other studies, “Olivenol and AlleOne Effect in Early Parkinson's Disease (“OLIVE-PD”) and “Olivenol and AlleOne Effect in Already Treated Parkinson's Disease Patients” (“AD-OLIVE-PD”). OLI-PD has three phases: (1) the two weeks prior to initiating Olivenol Plus administration; (2) four weeks of Olivenol Plus administration; and (3) two weeks as in phase (1).

OLIVE-PD inclusion criteria: recent onset PD (diagnosed according to EFNS-guideline criteria), Hoehn and Yahr stage 1 or 2, no invalidating tremor or freezing, signed informed consent.

OLIVE-PD exclusion criteria: subject below 18 years of age or above 80, rasagiline contraindication, PD therapy in the month prior to study inclusion (with the exception of possible L-dopa administration), gastrointestinal disease that may interfere with intestinal absorption, severe somatic or psychiatric disorders, antidepressant medication, impairments that may interfere with the neurological examination, pregnancy, postpartum period and breastfeeding.

AD-OLIVE-PD inclusion criteria: PD diagnosis, stable medication in the month prior to inclusion, informed consent.

AD-OLIVE-PD exclusion criteria: subject below 18 years of age or above 80, severe somatic or psychiatric disorders, impairments that may interfere with the neurological examination, pregnancy, postpartum period and breastfeeding.

OLI-PD inclusion criteria: inclusion in OLIVE-PD/AD-OLIVE-PD, informed consent.

OLI-PD exclusion criteria: administration of AlleOne™ in OLIVE-PD/AD-OLIVE-PD.

The subjects are evaluated as follows:

VISIT 0 (study inclusion/baseline): history; neurological evaluation (including Unified Parkinson Disease Rating Scale/UPDRS, Non-motor Symptoms Parkinson Disease scale/NMS-PD, Hamilton-Anxiety Rating Scale/Ham-ARS and Montreal Cognitive Assessment/MoCA); blood tests (routine, plus cellular stress response and oxidative stress evaluation); urine tests; continuation of current therapy; standardized journal entry.

VISIT 1 (VISIT 0+13-18 days): as in VISIT 0, plus initiation of Olivenol Plus administration (two capsules, tid); and standardized journal entry.

VISIT 2 (VISIT 1+28-35 days): history; neurological evaluation (including UPDRS, NMS-PD scale, Hamilton-DRS, Ham-ARS, and MoCA); blood tests (as in VISIT 0); urine tests; continuation of current therapy; standardized journal entry.

END OF STUDY VISIT (VISIT 2+13-18 days): history; neurological evaluation (including UPDRS, NMS-PD scale, Hamilton-ARS, and MoCA); blood tests (as before); urine tests; continuation of current therapy.

### ABBREVIATIONS USED

UPDRS—Unified Parkinson's Disease Rating Scale;

NMS-PD—Non-Motor System Assessment Scale for Parkinson's Disease (see Chaudhuri et al. (2006), The Lancet 5(3):235-245);

Hamilton-ARS or Ham-ARS—Hamilton Anxiety Rating Scale (see Hamilton (1959) Br. J. Med. Psychol. 32:50-55); and

MoCA—Montreal Cognitive Assessment Test (developed by Dr. Ziad Nasreddine, 1996).

Interim results at VISIT 2 for five patients are presented in Table 3, below. At the conclusion of the study, it is expected that the results will confirm the efficacy of the present invention in treating Parkinson's patients, particularly early stage Parkinson's patients.

TABLE 3

| Subject | VISIT 0 | VISIT 1 | VISIT 2 |
|---|---|---|---|
| SV | UPDRS: 29 | UPDRS: 30 | UPDRS: 32 |
| (48 yo, F) | MoCA: 28 | MoCA: 28 | MoCA: 28 |
| | NMS-PD: 88 | NMS-PD: 89 | NMS-PD: 64 |
| | Ham-ARS: 18 | Ham-ARS: 18 | Ham-ARS: 15 |
| RR | UPDRS: 21 | UPDRS: 21 | UPDRS: 20 |
| (72 yo, F) | MoCA: 30 | MoCA: 30 | MoCA: 29 |
| | NMS-PD: 18 | NMS-PD: 18 | NMS-PD: 14 |
| | Ham-ARS: 8 | Ham-ARS: 8 | Ham-ARS: 7 |
| TL | UPDRS: 28 | UPDRS: 21 | UPDRS: 41 |
| (46 yo, F) | MoCA: 27 | MoCA: 30 | MoCA: 28 |
| | NMS-PD: 12 | NMS-PD: 18 | NMS-PD: 13 |
| | Ham-ARS: 6 | Ham-ARS: 8 | Ham-ARS: 19 |
| BF | UPDRS: 18 | UPDRS: 18 | UPDRS: 23 |
| (36 yo, M) | MoCA: 29 | MoCA: 29 | MoCA: 30 |
| | NMS-PD: 5 | NMS-PD: 5 | NMS-PD: 4 |
| | Ham-ARS: 7 | Ham-ARS: 7 | Ham-ARS: 5 |
| CC | UPDRS: 36 | UPDRS: 36 | UPDRS: 47 |
| (37 yo, M) | MoCA: 26 | MoCA: 26 | MoCA: 26 |
| | NMS-PD: 11 | NMS-PD: 11 | NMS-PD: 15 |
| | Ham-ARS: 7 | Ham-ARS: 7 | Ham-ARS: 6 |

I claim:

1. A method for treating a subject suffering from early stage Parkinson's disease, comprising administering to the subject a therapeutically effective amount of a pharmaceutical formulation that comprises:
an olive polyphenol composition containing hydroxytyrosol and at least one additional olive polyphenol, wherein the hydroxytyrosol represents about 40 wt. % to about 90 wt. % of the olive polyphenol composition,
and wherein the therapeutically effective amount provides a daily dose of the olive polyphenol composition in the range of 30 mg to about 2500 mg.

2. The method of claim 1, wherein the subject suffers from idiopathic Parkinson's disease.

3. The method of claim 1, wherein the subject suffers from secondary parkinsonism.

4. The method of claim 1, wherein the hydroxytyrosol represents about 40 wt. % to about 60 wt. % of the olive polyphenol composition.

5. The method of claim 4, wherein the hydroxytyrosol represents about 42 wt. % to about 50 wt. % of the olive polyphenol composition.

6. The method of claim 1, wherein the pharmaceutical formulation is orally administered to the subject.

7. The method of claim 1, wherein the therapeutically effective amount provides a daily dose of the olive polyphenol composition in the range of 35 mg to about 1000 mg.

8. The method of claim 7, wherein the therapeutically effective amount provides a daily dose of the olive polyphenol composition in the range of 35 mg to about 300 mg.

9. The method of claim 1, wherein the pharmaceutical formulation further includes a carrier, and the olive polyphenol composition represents about 5 wt. % to about 50 wt. % of the formulation.

10. The method of claim 8, wherein the olive polyphenol composition represents about 10 wt. % to about 30 wt. % of the formulation.

11. The method of claim 1, wherein the pharmaceutical formulation is in the form of a liquid.

12. The method of claim 1, wherein the pharmaceutical formulation is in the form of a tablet or capsule.

13. The method of claim 1, wherein the pharmaceutical formulation is in the form of a dry powder.

14. The method of claim 1, wherein the pharmaceutical formulation comprises a unit dosage form.

15. The method of claim 1, wherein the pharmaceutical formulation provides for controlled release of the olive polyphenol composition.

16. The method of claim 15, wherein the controlled release is sustained release.

17. The method of claim 1, wherein the pharmaceutical formulation is administered one to about six times daily.

18. The method of claim 17, wherein the pharmaceutical formulation is administered two to about six times daily.

19. The method of claim 1, wherein the pharmaceutical formulation further includes an additional active agent.

20. The method of claim 19, wherein the additional active agent is an anti-parkinsonism drug.

21. The method of claim 20, wherein the anti-parkinsonism drug is selected from L-dopa, dopamine receptor agonists, amantadine hydrochloride, monoamine oxidase B inhibitors, catechol-O-methyltransferase inhibitors; and antimuscarinic agents.

22. The method of claim 1, wherein the at least one additional olive polyphenol comprises oleuropein.

23. The method of claim 22, wherein the oleuropein is present such that the weight ratio of hydroxytyrosol to oleuropein in the olive polyphenol composition is in the range of about 1:1 to about 100:1.

24. The method of claim 23, wherein the weight ratio of hydroxytyrosol to oleuropein in the olive polyphenol composition is in the range of about 1:1 to about 9:1.

25. A method for reducing the dosage of an anti-parkinsonism drug in the treatment of a subject suffering from early stage Parkinson's disease, the method comprising co-administering with the anti-parkinsonism drug a therapeutically effective amount of a pharmaceutical formulation that comprises:
an olive polyphenol composition containing hydroxytyrosol and at least one other olive polyphenol, wherein the hydroxytyrosol represents about 40 wt. % to about 90 wt. % of the olive polyphenol composition,
and wherein the therapeutically effective amount provides a daily dose of the olive polyphenol composition in the range of 30 mg to about 2500 mg.

26. A formulation for the treatment of early stage Parkinson's disease, comprising:
a therapeutically effective amount of an olive polyphenol composition containing hydroxytyrosol and at least one other olive polyphenol, wherein the hydroxytyrosol represents about 40 wt. % to about 90 wt. % of the olive polyphenol composition; and
a therapeutically effective amount of an antiparkinsonism drug selected from L-dopa, dopamine receptor agonists, amantadine hydrochloride, monoamine oxidase B inhibitors, catechol-O-methyltransferase inhibitors; and antimuscarinic agents.

27. A method for treating a subject suffering from Parkinson's disease, comprising administering to the subject a therapeutically effective amount of a pharmaceutical formulation that comprises:
an olive polyphenol composition containing hydroxytyrosol and at least one additional olive polyphenol, wherein the hydroxytyrosol represents about 40 wt. % to about 90 wt. % of the olive polyphenol composition,
wherein the at least one additional olive polyphenol comprises oleuropein, and the weight ratio of hydroxytyrosol to oleuropein in the olive polyphenol composition is in the range of about 1:1 to about 100:1.
and further wherein the therapeutically effective amount provides a daily dose of the olive polyphenol composition in the range of 30 mg to about 2500 mg.

26. The method of claim 25, wherein the weight ratio of hydroxytyrosol to oleuropein in the olive polyphenol composition is in the range of about 1:1 to about 9:1.

28. The method of claim 27, wherein the pharmaceutical formulation further includes an additional active agent.

29. The method of claim 28, wherein the additional active agent is an anti-parkinsonism drug.

30. The method of claim 29, wherein the anti-parkinsonism drug is selected from L-dopa, dopamine receptor agonists, amantadine hydrochloride, monoamine oxidase B inhibitors, catechol-O-methyltransferase inhibitors; and antimuscarinic agents.
